(19) United States
(12) Patent Application Publication
(10) Pub. No.: US 2006/0251209 A1
(43) Pub. Date: Nov. 9, 2006

(54) Energy sensitive x-ray system and method for material discrimination and object classification

(75) Inventors: J. Erie Tkaczyk, Delanson, NY (US); William Macomber Leue, Albany, NY (US); Deborah Joy Walter, Burnt Hills, NY (US); Yangfeng Du, Rexford, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/122,874

(22) Filed: May 6, 2005

(51) Int. Cl. H05G 1/60; A61B 6/00; G01N 23/00; G21K 1/12

(52) U.S. Cl. 378/5

(57) ABSTRACT

A system and method for classifying whether an object within an enclosed article is a threat. The system includes an acquisition subsystem, a reconstruction subsystem, an energy discrimination subsystem, and a classification subsystem. The acquisition subsystem communicates projection data to the reconstruction subsystem. The energy discrimination subsystem, which utilizes energy spectra at two distinct ranges, uses the projection data to ascertain the compositional make up of the object of interest

### BACKGROUND

The invention relates generally to the detection and classification of objects located within articles. More particularly, the invention relates to the classification of harmful objects, such as, for example, contraband.

There has always been, and there continues to be, a demand for heightened security surrounding various communication and transportation avenues. For example, metal detectors and x-ray machines are standard security devices employed at airports for screening passengers and their carry-on luggage. The United States Postal Service also employs x-ray technology for screening parcels.

The capability for automatically screening luggage in an efficient and cost-effective manner is currently non-existent. The screening systems currently in place record false positives at higher than desirable rates. The high number of false positives forces alternative follow-on inspections, such as trace detection or manual inspection of the luggage, thereby increasing the average screening time per bag substantially. There remains a need for a high-throughput (e.g., at least one thousand scanned checked bags per hour) automatic screening system for ascertaining whether a piece of luggage or a mail parcel contains an object which may be harmful, such as, for example, an explosive device or material.

Further, there remains a demand for improved medical diagnostic capabilities, specifically improved techniques for ascertaining whether harmful and potentially life-threatening objects are within a body. For example, an improved technique for ascertaining whether and to what extent plaque is building up within arteries is of importance.

### SUMMARY

The invention described herein is directed to a system and a method for ascertaining whether an object located within a closed article, such as within luggage or within the human body, is a harmful object, such as an explosive device or a build up of plaque within an artery.

One aspect of the invention is an object detecting system. The system includes an acquisition subsystem for acquiring projection data on the object, an energy discriminating subsystem for obtaining attenuation information on the object at two distinct energy spectra, and a reconstruction subsystem for rendering image data of the object from the projection data.

Another aspect of the invention is a method for detecting an object. The method includes the steps of scanning an article containing the object, obtaining projection data from the scanning, and obtaining vector data from the projection data for each voxel of the object. The vector data includes at least two distinct scalar measurements.

Another aspect of the invention is a method for classifying an object. The method includes the steps of scanning an article containing the object, obtaining projection data from the scanning, obtaining vector data from the projection data for each voxel of the object, and classifying the object based on the vector data. The vector data includes at least two distinct scalar measurements.

These and other advantages and features will be more readily understood from the following detailed description of preferred embodiments of the invention that is provided in connection with the accompanying drawings.

### BRIEF DESCRIPTION OF THE DRAWINGS

FIG. 1 is a schematic view of an object classification system in accordance with an exemplary embodiment of the invention.

FIG. 2 is a schematic representation of attenuation values taken in separate energy ranges by the reconstruction subsystem of FIG. 1.

FIG. 3 is a schematic representation of the density of an object M based upon the density of two base objects A and B obtained from the attenuation values of FIG. 2.

FIG. 4 is a schematic representation of an orthogonal decomposition using the attenuation values of FIG. 2.

FIG. 5 is a schematic representation of the density of an object M based upon the density of two base objects A and B obtained from the orthogonal decomposition of FIG. 4.

FIG. 6 is a schematic representation of the classification of objects M1 and M2 in accordance with an exemplary embodiment of the invention.

FIG. 7 illustrates a process for obtaining a classification of an object in accordance with an exemplary embodiment of the invention.

### DETAILED DESCRIPTION OF PREFERRED EMBODIMENTS

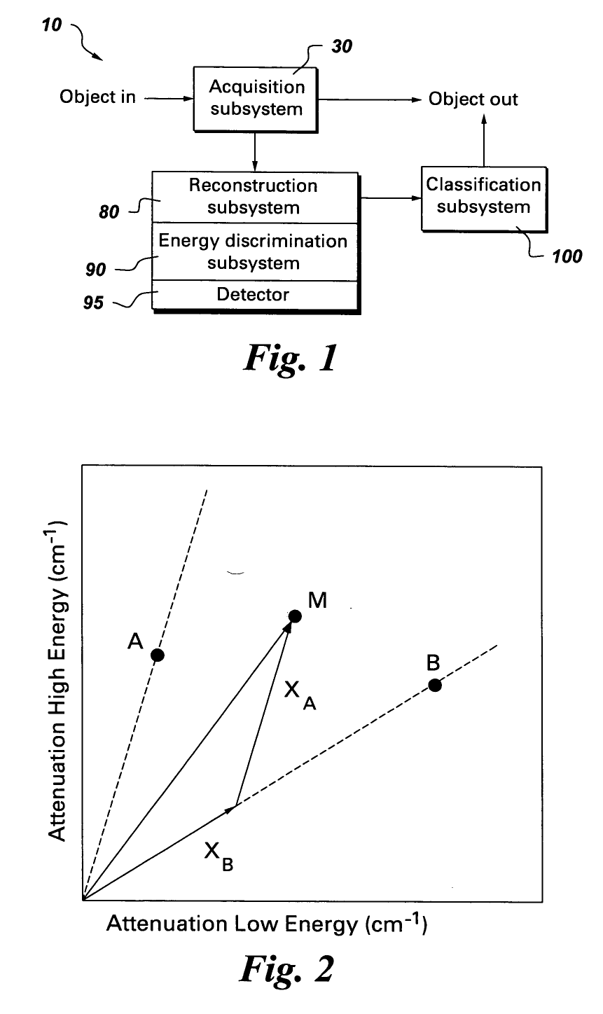

With reference to FIG. 1, an object classifying system 10 is schematically shown. The object classifying system 10 includes an acquisition subsystem 30, a reconstruction subsystem 80, an energy discrimination subsystem 90, and a classification subsystem 100. The acquisition subsystem 30 may be adapted to accommodate a high throughput of articles, for example, screening of upwards of one thousand individual pieces of luggage within a one hour time period to locate contraband (i.e., explosive materials or other dangerous substances generally prohibited from transportation within airliners or from mailing), with a high detection rate and a tolerable number of false positives. Alternatively, the acquisition subsystem 30 may be adapted to accommodate a low throughput of articles, for example, scanning a patient on a table to look for a health-related anomaly.

The acquisition subsystem 30 may take the form of numerous embodiments. One embodiment of the acquisition subsystem 30 is a computed tomography (CT) scanner, which includes a source, a target and a detector. For example, the acquisition subsystem 30 may incorporate a 5^{th} generation CT scanner, one having stationary radiation sources and detectors. The 5^{th} generation CT scanner includes a vacuum housing chamber that generates an electron beam. The electron beam is swept by magnetic fields and scans an arc-shaped target. Upon being struck by the electron beam, which typically scans 210 degrees or so in about 50 ms, the target emits a moving fan-like beam of x-rays that passes through a region of the article lying atop a conveyor belt, and then registers upon a detector array located diametrically opposite. The detector array measures intensity profiles of transmitted x-ray beams, allowing generation of view data, or projection data, that is then communicated to the reconstruction subsystem 80.

Alternatively, the acquisition subsystem 30 may incorporate a CT scanner having stationary radiation sources and detectors. Such a CT scanner may include a source ring including distributed electron field emission devices. Such a CT scanner further includes a detector ring adjacent to the source ring. The detector ring may be offset from the source ring. It should be appreciated, however, that “adjacent to” should be interpreted in this context to mean the detector ring is offset from, contiguous with, concentric with, coupled with, abutting, or otherwise in approximation with the source ring.

The acquisition subsystem 30 may include an energy discriminating function, including a detector 95 (FIG. 1), to allow the use of a multi-energy CT approach. An energy discriminating function utilizes information regarding the attenuation of x-rays of different energies penetrating the object of interest. Typically, this information can be obtained either by acquiring projection data with two or more different source spectral profiles (achieved by varying source kvp voltage, source filtration, or a combination of the two) or by achieving a spectral decomposition of a single source spectrum in the detector elements. For example, the acquisition subsystem 30 may include at least one detector for detecting x-rays from at least two different incident x-ray energy spectra. Alternatively, the acquisition subsystem 30 may include either an energy discriminating detector adapted to acquire energy sensitive measurements in the photon counting mode or an energy discriminating detector that includes an assembly of two or more x-ray attenuating materials, the signals from which can be processed in either a photon counting or a charge integration mode. The projection data pertaining to the energy discriminating function is forwarded, along with other projection data, to the reconstruction subsystem 80, and also on to the energy discriminating subsystem 90.

A conventional CT scanner scans with a source at a particular high voltage, such as, for example, between 120 and 140 keV. Such CT scanners give a broad spectrum of energy data. As an x-ray penetrates an object, attenuation occurs. Attenuation is greater for lower energy signals, which causes beam hardening. Beam hardening complicates the analysis of an object by overestimating its density, thereby distorting the image produced. Conventional reconstruction subsystems take the energy data and provide a scalar number. Thus, using conventional scanners and reconstruction subsystems, two objects having different atomic numbers and different densities may have a resulting scalar number that is similar. A CT scanner capable of dual energy scanning, such as the acquisition subsystem 30, will allow computation of a pair of data sets. One of the data sets can be directed to mass density, while the other can be directed to an average Z value (atomic number) for items within the article being scanned. Alternatively, one of the data sets can be directed to low energy attenuation (about 80 keV), while the other data set can be directed to high energy attenuation (about 120 keV or higher).

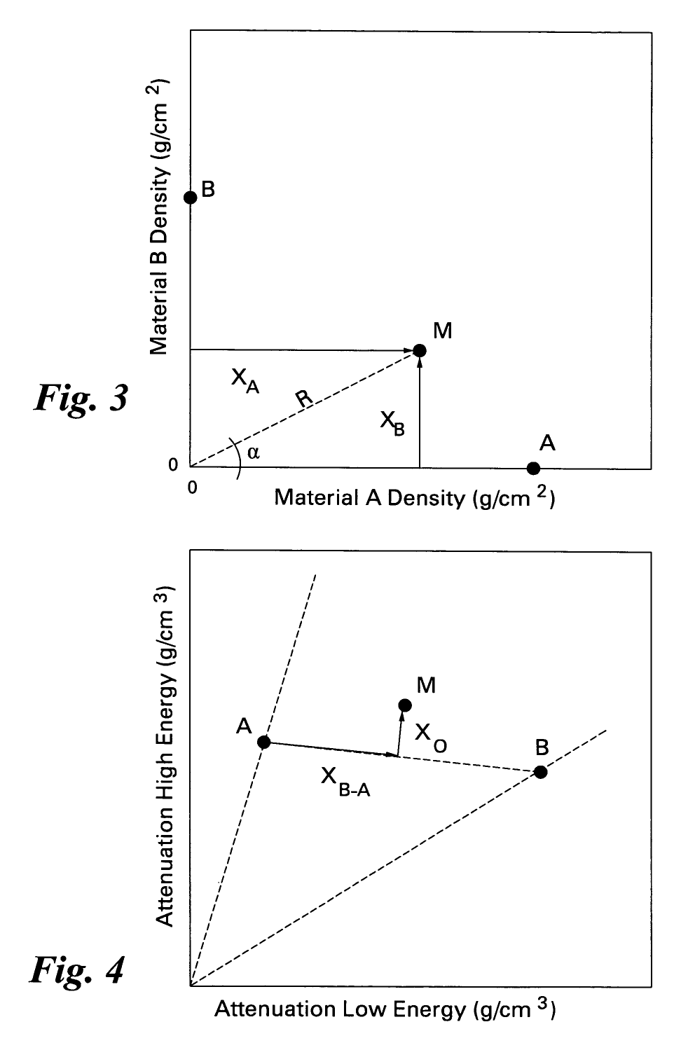

With specific reference to FIG. 2, there is shown a graph plotting high energy attenuation against low energy attenuation. Two objects A and B, having a known compositional makeup, are plotted on the graph. Each of the objects A and B should have a compositional makeup that is consistent with the detection and classification goal of the object classification system 10. Objects A and B may respectively comprise, for example, aluminum and acrylic. The detection vectors of the objects A and B are located as two distinct points in the detection vector space. The positions of known objects A and B in the attenuation map of FIG. 2 are specifically dependent on the system's energy response characteristics, and these positions represent a calibration of the system's energy response. A third object M, having an unknown compositional makeup, also is plotted on the graph. The position of object M relative to calibration objects A and B in the FIG. 2 graph is determined by the vectors X_{A} and X_{B}. Vector X_{B} is parallel to the line from 0, 0 to the position of B on the graph, while vector X_{A} is parallel to the line from 0, 0 to the position of A on the graph. FIG. 3 is an alternate plot showing the density of the object M as plotted against the densities of the objects A and B. The relative position of M with respect to X_{A} and X_{B}, in FIG. 3 is independent of the system's energy response characteristics and is an exemplary representation of M relative to that in FIG. 2.

The angle α of the line in FIG. 3 connecting 0,0 to the position of M on the graph is representative of the effective atomic number of the material M. Further, the radial distance R from 0,0 to the position of M is representative of the effective density of M. The effective atomic number, radial distance R, and the angle α in this calibration density space shown in FIG. 3 is a useful representation of the material M for the purposes of classifying its composition.

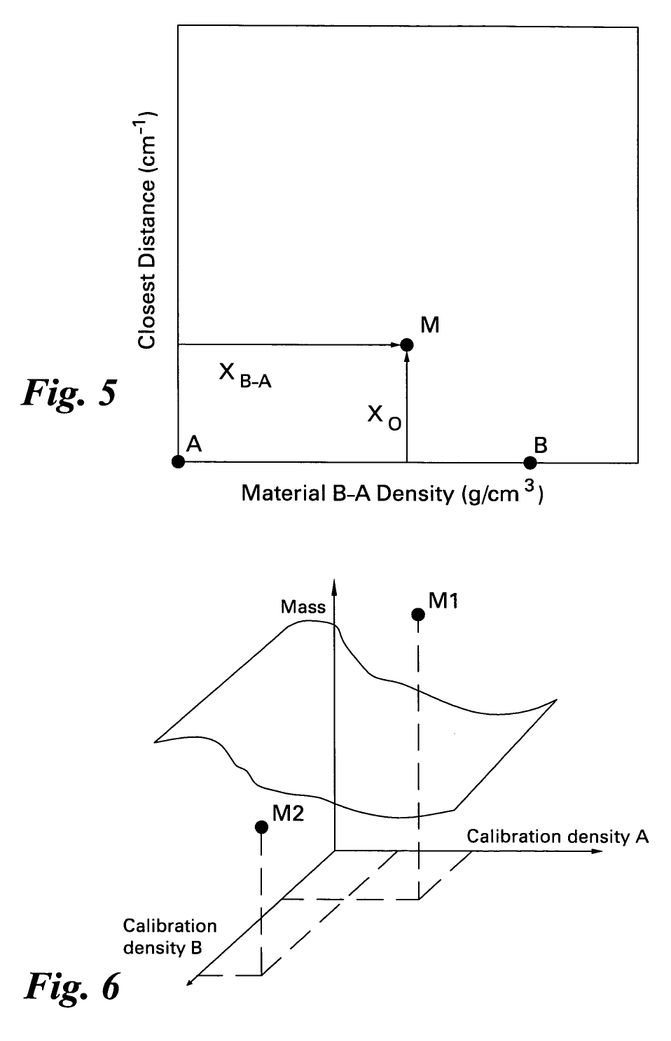

With reference to FIG. 4, there is shown a line X_{B-A} extending between the two distinct points in the detection vector space identifying the detection vectors of objects A and B. For any third material, such as object M, the projection of the material detector vector (the vector product) onto the line X_{B-A} gives the equivalent fraction of he mixture of these two objects A and B. A second scalar number X₀ is obtained by taking the shortest distance from the material detection vector M to the line X_{B-A}. The scalar numbers are a reduced dimensionality of the original detection vector. As such, the scalar numbers may be subject to a look up table or algorithmic calculation for identification and classification purposes. Using the plots as shown in FIG. 4, a density of object M can be mapped on the graph of FIG. 5. As shown therein, objects A and B are on the x-axis of the density differential between objects A and B, and object M is a distance X_{B-A} from the y-axis (distance) and a distance X₀ from the x-axis.

Referring to FIGS. 2-5, as an example object A is iodine and object B is water. For vascular imaging, mixtures of iodine and water are useful in highlighting the vascular structure. The detection vector for other materials, such as those containing calcium (calcified plaque) would present itself somewhere between the two detection vectors for objects A and B. The shortest distance scalar X₀ identifies the calcified plaque by how much it differs from the iodine-water mixture. This data then can be used either in segmentation algorithms or in color maps to provide visual distinction between the calcified plaque and the iodine-water mixture within the vascular structure.

FIG. 6 is a visual representation of the plotting of two objects M1 and M2 against three parameters, specifically mass, density calibration material A, and density calibration material B. By plotting the objects M1 and M2 against three parameters, the objects M1 and M2 can be plotted against a threshold surface. A threshold surface is a surface that defines, depending upon where an object plots on the three-dimensional graph relative to the threshold surface, whether the object is one that may be considered a threat and one that may be considered benign. For example, objects that plot at a point sufficiently high along the axis delineating mass are above the threshold surface, such as M2, and thus are considered a threat. Conversely, objects that plot at a point beneath the threshold surface, such as M1, are considered benign. Alternatively, for a threshold surface that is closed, it is readily ascertained whether an object point is within or without the surface.

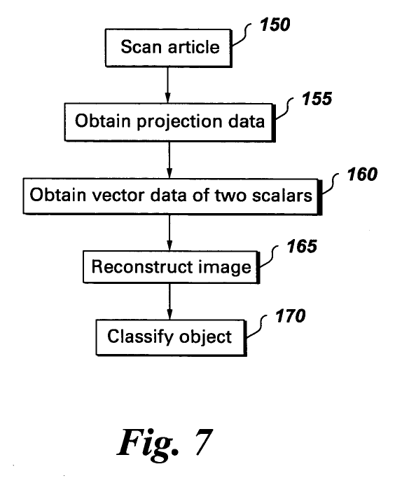

In practice, and in reference with FIGS. 1 and 7, next will be described a process for classifying an object within a scanned article through the use of the object classification system 10. In Step 150, an article is scanned by the acquisition subsystem 30. As noted previously, the acquisition subsystem 30 may include a CT scanner. Through the process of scanning, projection data is obtained at Step 155. On average, each article scanned by the acquisition subsystem 30 will consist of 10,000 to 20,000 voxels. By scanning with the acquisition subsystem 30, which includes an energy discriminating function, at Step 160 a projection set of vector data may be obtained comprising a set of two scalars, namely low energy attenuation and high energy attenuation. Alternatively, at Step 160 the two scalars obtained may be density of calibration material A and density of calibration material B in accord with the transformation from FIG. 2 to FIG. 3. At Step 165, the projection data obtained at Step 155 is forward to the reconstruction subsystem 80 to reconstruct from the projection data images of the article being scanned. It should be appreciated that Step 160 may be performed either prior to or subsequent to Step 165. One can perform Step 160 subsequent to Step 165 by operating on the set of image data obtained from the reconstruction of the projection data set for each vector component.

Finally, at Step 170, either the projection data or the image data based upon the projection data and obtained from the reconstruction subsystem 80 at Step 165 is forwarded to the classification subsystem 100 to identify the object within the scanned article and classify either as a threat or as a non-threat. Individual components within the total volume of the scanned article may be segmented from the volume, as is known in the art. Individual components are identified as a subset of the total number of voxels. The object and components therein are assigned a mass scalar value by summing the density over the voxels contained within the object. Other scalar quantities are similarly obtained by operating on the vector values for all voxels within the object and its components. A threshold map such as shown in FIG. 6 is applied to classify the object and/or components within the object.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed as new and desired to be protected by Letters Patent of the United States is:

1. An object detecting system, comprising:
an acquisition subsystem for acquiring projection data on the object;
an energy discriminating subsystem for obtaining attenuation information on the object at two distinct energy spectra; and
a reconstruction subsystem for rendering image data of the object from the projection data.

2. The object detecting system of claim 1, wherein the acquisition subsystem comprises a computed tomography machine.

3. The object detecting system of claim 1, wherein said energy discriminating subsystem comprises at least one detector for detecting x-rays from at least two different incident x-ray energy spectra.

4. The object detecting system of claim 1, wherein said energy discriminating subsystem comprises an energy discriminating detector adapted to acquire energy sensitive measurements in the photon counting mode.

5. The object detecting system of claim 1, wherein said energy discriminating subsystem comprises an energy discriminating detector that includes an assembly of two or more x-ray attenuating materials, the signals from which can be processed in either a photon counting or a charge integration mode.

6. The object detecting system of claim 1, wherein said energy discriminating subsystem is adapted to determine the compositional makeup of the object by comparison with at least two other objects whose compositional makeup is known.

7. The object detecting system of claim 1, further comprising a classification subsystem for determining whether the object is a threat or whether individual components within the object are a threat.

8. The object detecting system of claim 1, wherein said acquisition subsystem is adapted to scan for contraband objects.

9. The object detecting system of claim 1, wherein said acquisition subsystem is adapted to scan for health-related anomalies.

10. The object detecting system of claim 9, wherein said health-related anomalies comprises calcified plaque.

11. A method for detecting an object, comprising:
scanning an article containing the object;
obtaining projection data from said scanning; and
obtaining vector data from the projection data for each voxel of the object, wherein the vector data includes at least two distinct scalar measurements.

12. The method of claim 11, wherein the at least two distinct scalar measurements are chosen from a group consisting of mass, density, effective atomic number, high energy attenuation, low energy attenuation, density of a known calibration material, angle in the calibration density space, and radial position in the calibration density space.

13. The method of claim 11, further comprising reconstructing image data from the projection data.

14. The method of claim 1 1, wherein said obtaining projection data comprises obtaining attenuation data from at least two different incident x-ray energy spectra.

15. A method for classifying an object or components therein, comprising:
scanning an article containing the object;
obtaining projection data from said scanning;
obtaining vector data from the projection data for each voxel of the object, wherein the vector data includes at least two distinct scalar measurements; and
classifying the object or individual components within the object based on the vector data.

16. The method of claim 15, wherein the at least two distinct scalar measurements are chosen from a group consisting of mass, density, effective atomic number, high energy attenuation, low energy attenuation, density of a known calibration material, angle in the calibration density space, and radial position in the calibration density space.

17. The method of claim 15, further comprising reconstructing image data from the projection data.

18. The method of claim 15, wherein said obtaining projection data comprises obtaining attenuation data from at least two different incident x-ray energy spectra.

19. The method of claim 18, wherein said classifying comprises combining the energy-specific attenuation data along with other scalar data derived from projection data for each voxel of the object.

20. The method of claim 19, wherein said classifying further comprises comparing the combined data for the object against a threshold surface to ascertain whether the object is a threat.
